(19) United States
(12) Patent Application Publication
(10) Pub. No.: US 2021/0319876 A1
(43) Pub. Date: Oct. 14, 2021

(54) COMPUTER IMPLEMENTED METHOD, A SYSTEM AND COMPUTER PROGRAM FOR DETERMINING PERSONALILZED PARAMETERS FOR A USER

(71) Applicant: Koa Health B.V., Barcelona (ES)

(72) Inventors: Mohammed Khwaja, Madrid (ES); Aleksandar Matic, Lloret de Mar (ES)

(21) Appl. No.: 17/354,458

(22) Filed: Jun. 22, 2021

Related U.S. Application Data
(63) Continuation-in-part of application No. PCT/EP2019/071070, filed on Aug. 6, 2019.

(30) Foreign Application Priority Data
Dec. 27, 2018 (EP) 18382999.3
Aug. 6, 2019 (EP) PCT/EP2019/071070

(51) Int. Cl. G16H 20/70; G16H 50/20; G16H 10/20; G16H 40/67

(52) U.S. Cl. CPC G16H 20/70; G16H 40/67; G16H 10/20; G16H 50/20

(57) ABSTRACT

Method, system and computer program for determining personalized parameters for a user. The method comprises providing a first vector of personal characteristics based on received first data, a second vector of behavior and activity characteristics based on received second data, and a third vector of wellbeing measures based on received third data. Exhibited personal characteristics and the first vector is also calculated. A reference group for the user is created and a similarity measure between the user and the reference group is implemented to identify which of said users has more characteristics in common with the user. An optimal behavior and activity distribution vector can be determined from the most similar users of said reference group. The range of behaviors and activities that are good or bad for the user can be also determined.

### CROSS REFERENCE TO RELATED APPLICATION

This application is filed under 35 U.S.C. § 111(a) and is based on and hereby claims priority under 35 U.S.C. § 120 and § 365(c) from International Application No. PCT/EP2019/071070, filed on Aug. 6, 2019, and published as WO 2020/135936 A1 on Jul. 2, 2020, which in turn claims priority from European Application No. EP18382999.3, filed in the European Patent Office on Dec. 27, 2018. This application is a continuation-in-part of International Application No. PCT/EP2019/071070, which is a continuation of European Application No. EP18382999.3. International Application No. PCT/EP2019/071070 is pending as of the filing date of this application, and the United States is an elected state in International Application No. PCT/EP2019/071070. This application claims the benefit under 35 U.S.C. § 119 from European Application No. EP18382999.3. The disclosure of each of the foregoing documents is incorporated herein by reference.

### TECHNICAL FIELD

Present invention relates generally to computing methods and systems. In particular, the invention relates to a method, system and computer programs for determining personalized parameters for a user. In this sense, the parameters can be used to provide to the user personalized recommendations about lifestyle modifications, such as changes to the user's normal activities and behavior

The present application has received funding from the European Union's Horizon 2020 research and innovation program under the Marie Sktodowska-Curie grant agreement No 722561.

### BACKGROUND

U.S. Patent Publication 2017/0132395 provides a connected digital therapeutics navigation guidance system for lifestyle modification and disease prevention. The system includes wearable user devices to detect user data relating to health or fitness parameters, to receive user input data relating to the health or fitness parameters through a user interface, and to request a health or fitness routine from a server, which provides the wearable user device with a routine that can be performed by the user of the wearable user device.

U.S. Patent Publication 2012/0059785 relates to a content recommendation method and system based on psychological factors from a user profile. The system includes: (a) a website for downloads that is accessible for the user, where said site comprises means configured for the detection of his consumption profile; (b) a psychographics driver configured to calculate his profile in the five super traits of the Big Five model, said profile being stored in a first database, said data being accessible for the current query and subsequent queries; and (c) means configured to cross-match the psychological profile stored in the first database and the data contained in a second content database; all such that access preferably to those contents which best adapt to the calculated psychological profile is granted to the user.

U.S. Pat. No. 7,457,768 describes a new recommendation technique that can be seen as a hybrid between memory-based and model-based collaborative filtering techniques. Using personality diagnosis, all data may be maintained throughout the processes, new data can be added incrementally, and predictions have meaningful probabilistic semantics. Each entity's (e.g., user's) reported attributes (e.g., item ratings or preferences) may be interpreted as a manifestation of its underlying personality type. Personality type may be encoded simply as a vector of the entity's (e.g., user's) “true” values (e.g., ratings) for attributes (e.g., items) in the database. It may be assumed that entities (e.g., users) report values (e.g., ratings) with a distributed (e.g., Gaussian) error. Given an active entity's (e.g., user's) known attribute values (e.g., item ratings), the probability that they have the same personality type as every other entity (e.g., user) may be determined. Then, the probability that they will have a given value (e.g., rating) for a valueless (e.g., unrated) attribute (e.g., item) may then be determined based on the entity's (e.g., user's) personality type. The probabilistic determinations may be used to determine expected value of information. Such an expected value of information could be used in at least two ways. First, an interactive recommender could use the expected value of information to favorably order queries for attribute values (e.g., item ratings), thereby mollifying what could otherwise be a tedious and frustrating process. Second, the expected value of information could be used to determine which entries of a database to prune or ignore, that is, which entries if removed would have a minimal effect of the accuracy of recommendations.

Chinese Patent No. 103309976-B discloses a method for improving the social recommendation efficiency based on user personality. The method comprises the steps: determining k commodities to be recommended to a target user; for each commodity m to be recommended, calculating the comprehensive recommendation scores of the good friends of the target user based on personalities and commodity preferences respectively, and selecting the friend with the highest score as a corresponding recommender of the commodity m; and notifying each recommender to recommend the corresponding commodity to the target user. According to the method, the proper recommender is selected according to the user personality features and the commodity preferences, so that the recommendation efficiency is improved.

The known collaborative filtering systems based on personality do not dynamically calculate personality from behaviors and compare this to static personality traits to form similarities.

New methods and systems for determining personalized parameters for a user are therefore sought that allow both known and unknown psychological constructs and personal characteristics to be incorporated.

### SUMMARY

A novel method for determining personalized parameters of a user involves identifying which of the users of a reference group are most similar to the user of the novel system in terms of their personal characteristics and their behavior and activity characteristics. First data is received regarding personal characteristics of the user of the system, and a first vector of personal characteristics of the user are determined based on the first data. Second data is received regarding behavior and activity characteristics of the user, and a second vector of behavior and activity characteristics of the user is determined based on the second data. Third data is received regarding subjective wellbeing measures of the user, and a third vector of subjective wellbeing measures of the user is received based on the third data. Exhibited personal characteristics of the user are determined using the first vector and the second vector. A miss-alignment parameter is determined that compares the personal characteristics of the user to the first vector of the user. A reference group for the user is identified that includes a plurality of users, each of whom has a higher third vector than that of the user and a lower miss-alignment parameter than that of the user. A similarity measure is determined between the user and the reference group, wherein the similarity measure identifies which of the users of the reference group are most similar to the user in terms of the personal characteristics and the behavior and activity characteristics.

In a first embodiment, an optimal behavior and activity distribution vector for the user is determined from the users of the reference group who are most similar to the user. The optimal behavior and activity distribution vector is then used to recommend behavior and activity modifications to the user. In a second embodiment, ranges of behavior and activities are determined that are good or bad for the user by training a machine learning model to predict a score of the subjective wellbeing measures using \vec{Δ}, which is calculated by generating all possible combinations of the second vector of behavior and activity characteristics, mapping the combinations to the exhibited personal characteristics of the user, and comparing the exhibited personal characteristics to the first vector of personal characteristics. The ranges of behavior and activities are then used to recommend behavior and activity modifications to the user.

A system for determining personalized parameters for a user includes a memory, a processor, a server and a sensor. The processor is configured to receive first, second and third data. The processor receives the first data regarding different personal characteristics of a user and determines a first vector of personal characteristics of the user of the system based on the first data. The processor receives the second data regarding behavior and activity characteristics of the user and determines a second vector of behavior and activity characteristics of the user based on the second data. The processor receives the third data regarding one or more subjective wellbeing measures of the user and determines a third vector of wellbeing measures of the user based on the third data. The processor determines exhibited personal characteristics of the user using the first vector and the second vector and determines a miss-alignment parameter between the exhibited personal characteristics and the first vector. The processor identifies a reference group for the user that includes a plurality of users, each of whom has a higher third vector than that of the user of the system and a lower miss-alignment parameter than that of the user of the system. The processor determines a similarity measure between the user and the reference group that identifies which of the users of the reference group are most similar to the user in terms of the personal characteristics and the behavior and activity characteristics. The processor determines from the users of the reference group who are most similar to the user an optimal behavior and activity distribution vector for the user and uses the optimal behavior and activity distribution vector to recommend behavior and activity modifications to the user.

Other embodiments and advantages are described in the detailed description below. This summary does not purport to define the invention. The invention is defined by the claims.

### BRIEF DESCRIPTION OF THE DRAWING

The accompanying drawings, where like numerals indicate like components, illustrate embodiments of the invention.

FIG. 1 schematically illustrates the different elements and/or tools that can be used for capturing the personal characteristics and the behaviors and activities of the user.

FIG. 2 schematically illustrates one embodiment for calculating the similarity measures between the user and other users within the reference group.

FIG. 3 schematically illustrates another embodiment for calculating the similarity measures between the user and other users within the reference group.

FIG. 4 schematically illustrates an embodiment for determining the range of behaviors and activities (actual lifestyle) that are good and bad for the user.

### DETAILED DESCRIPTION

Reference will now be made in detail to some embodiments of the invention, examples of which are illustrated in the accompanying drawings.

A method for determining personalized parameters for a user is implemented by a computer system that includes a memory and one or more processors. The method involves receiving three categories of data. First data is received regarding different personal characteristics of a user. Then a first vector of personal characteristics of the user is generated based on the first data. Second data is received regarding behavior and activity characteristics of the user. Then a second vector of behavior and activity characteristics of the user is generated based on the second data. Third data regarding one or more subjective wellbeing (SWB) measures of the user is received. Then a third vector of wellbeing measures of the user is generated based on the received third data.

Then, the computer system calculates exhibited personal characteristics of the user using the first and second vectors. The computer system is also used to calculate a miss-alignment parameter between the calculated exhibited personal characteristics and the first vector of the user. The computer system creates a reference group for the user that includes a plurality of users having a higher third vector and lower miss-alignment parameter than the user. A similarity measure between the user and the reference group is determined to identify which of the plurality of users within the reference group have more characteristics in common with the user.

In one embodiment, the computing system determines a user's optimal behavior and activity distribution vector from the most similar users of the reference group. The determined user's optimal behavior and activity distribution vector are used to recommend behavior and activity modifications for the user.

In another embodiment, the computing system determines a range of behaviors and activities that are good or bad for the user by training a machine learning model that predicts a score of the subjective wellbeing measures using \vec{Δ}, wherein \vec{Δ} is calculated by: generating all possible combinations of the second vector of behavior and activity characteristics, mapping the combinations to the calculated exhibited personal characteristics of the user, and comparing the calculated exhibited personal characteristics with the first vector of personal characteristics.

In another embodiment, the exhibited personal characteristics of the user are calculated by calculating a user's behavior and activity distribution matrix (LDM) by concatenating the second vector with second vectors of other users stored in a database. A correlation matrix C is calculated by correlating the first vector with the second vector. A weight matrix is determined using the following equation W=LDM×C, where x is the matrix multiplication operator. The exhibited personal characteristics are calculated using the following equation:

$${\vec{PC}}_{exhibited}^{( j )} = {\vec{PC}}_{med} + {\vec{PC}}_{med} \cdot {\vec{w}}^{( j )} = {\vec{PC}}_{med} ( 1 + {\vec{w}}^{( j )} )$$

where \vec{PC}_{med} is the median of the first vector and (⋅) is the Hadamard product of the weights of the user with said median of the first vector.

In an embodiment, the similarity measure is calculated by comparing the first vector of the user with a first vector of each one of the plurality of users within the reference group using a cosine similarity measure. In this case, the first vector of each one of the plurality of users being calculated using personal characteristics of each user.

In another embodiment, the similarity measure is calculated by comparing the first vector of the user with calculated exhibited personal characteristics of each one of the plurality of users within the reference group using a cosine similarity measure. In this case, the exhibited personal characteristics of each one of the plurality of users are calculated using personal characteristics and behavior and activity characteristics of each user.

In one aspect, the first data is received from a server providing results of a personality survey implemented by the user. Optionally, the first data can be further inferred through knowledge of variables of the user including people from the same neighborhood or community and/or by using a clustering or machine learning algorithm on the variables. The second data is received as a vector quantifying a proportion of the different daily activities of the user, where each activity is assigned a specific value or a specific percentage.

In another aspect, the second data is received from a server providing a self-report of the user and/or from a passive sensor, mobile phone, wearable device or activity recognition system of the user. The third data can be received from a sensor that monitors activities or physiological signals of the user including body temperature, heart rate parameters, voice parameters and/or facial expressions.

Other embodiments of the invention involve a system and computer programs that include instructions embodied in a non-transitory computer readable medium. When executed by a processor, the instructions cause the processor to determine personalized parameters for a user. A novel method relies on the psychological principle that for an individual living life in line with “who he is” in terms of personal characteristics (such as personality, demographics, psychological profiles, biological characteristics, etc.) leads to an improved subjective wellbeing (SWB). There are individuals whose lifestyles (i.e., behavior and activities) are aligned with their personal characteristics and who have a high SWB, and there are other individuals whose lifestyles are not aligned with their personal characteristics and who have a low SWB. Assessing the “alignment” between behavior and activity characteristics and personal characteristics is performed using the concept of “exhibited personal characteristics”. Exhibited personal characteristics are determined by modeling personal characteristics based on the observed lifestyle, and then comparing the modeled exhibited personal characteristic with the actual one (known from another source, such as a survey, questionnaire, clustering method, belonging to specific demographics, etc.) aiming to reduce the gap between the two by modifying the lifestyle of the user.

The novel method dynamically models personal characteristics of a user based on the observed behavior and activities of the user. The method infers whether the user's lifestyle is aligned with his/her personal characteristics, and then recommends changes so that the user can live closer to an optimal lifestyle. For example, the optimal lifestyle is inferred through the analysis of characteristics of users with similar personal characteristics to the user who are positive role models with respect to the quality of life measured as a subjective wellbeing. Thus, the novel method can be used as a recommendation system for modifying time distribution of daily activities in one's life.

Present invention provides a method, and corresponding system, The novel method for determining personalized parameters of a user involves quantifying exhibited personal characteristics. In a first step, the actual personal characteristics of the user are captured or received. Then, the user's behavior and activities (actual lifestyle) and subjective wellbeing (SWB) indices/characteristics are captured or received. The exhibited personal characteristics are quantified, and a miss-alignment value/parameter is computed. A reference group is identified, and a recommendation is generated. The range of behavior and activities (actual lifestyle) that are good and bad for the user are determined, and an additional recommendation is generated and proposed to the user.

### Capturing Personal Characteristics

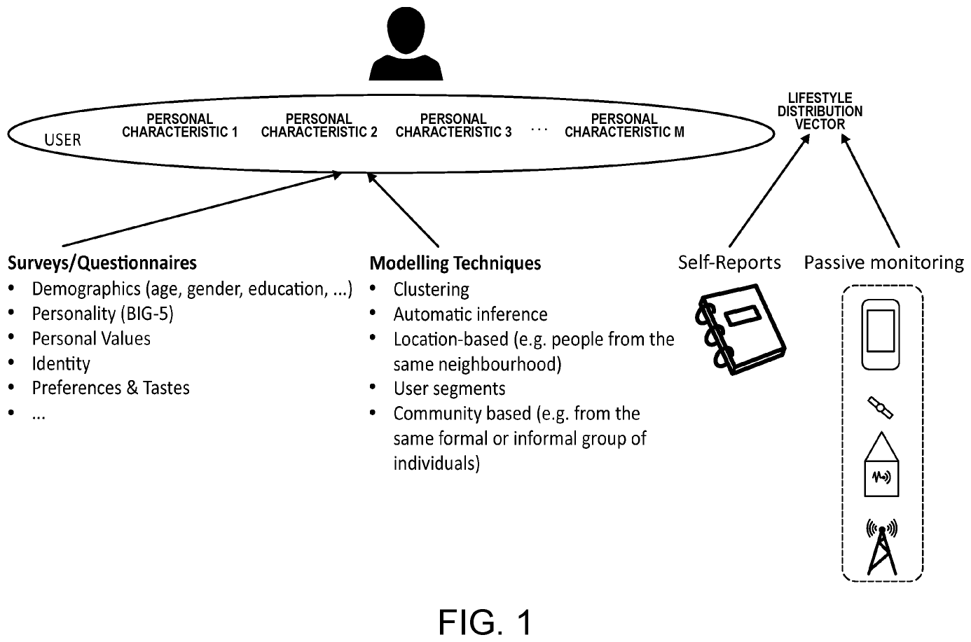

FIG. 1 illustrates how personal characteristics (also called the first data) can be self-reported through surveys or questionnaires using validated or non-validated inventories. For instance, personality traits can be acquired through asking the user to fill out the validated BIG-5 personality questionnaire. The questionnaire reveals the amounts by which the user exhibits the five components of personality or personal characteristics, which are extroversion, agreeableness, conscientiousness, emotional stability and intellect. Personal characteristics can also be inferred indirectly through knowledge of other variables or by using these variables with clustering or machine learning techniques based on such variables. The novel method can rely on one or more personal characteristics acquired in one or more ways.

Capturing the user's behavior, activities and SWB

Lifestyle represents a description of the user's typical activities and behavior in life. The user's typical activities and behavior can be expressed as a vector that quantifies the relative proportions of different activities, such as sleeping, driving, reading, watching TV, . . . −>23, 12, 31, 52, . . . where each activity is assigned a specific number of points (openly defined for a specific implementation) or a percentage (26%, 4%, 5%, 1%, . . . ) that defines the proportion of life in an observed period of time that the user spends on specific categories of activities.

Observing the user's behavior and activities can be conducted using typical self-reporting strategies, such as diaries, (daily) reconstruction methods, ecological momentary assessments, etc., aggregated over a specific period of time (such as a few weeks, months, years) to obtain the second vector (also called the lifestyle distribution vector (LDV)). Activities and behavior can also be captured using passive sensors, mobile phones, wearables, environmental sensors and/or activity recognition systems.

The lifestyle distribution vector (LDV) can combine activities acquired in a passive way or through self-report methods. On the other hand, subjective wellbeing (SWB) can be acquired once or multiple times in self-reported ways. Subjective wellbeing (SWB) includes one or a combination of the following wellbeing measures: an evaluative wellbeing score (life satisfaction), a hedonic wellbeing score (sense of pleasure), and a eudemonic wellbeing (sense of purpose) score. SWB can also be modeled through data acquired from passive monitoring of activities or physiological signals, such as body temperature, heart rate, heart rate variability, non-verbal voice analysis, video analysis of facial expressions, etc.

Thus, for each user j, three vectors are determined with the aforementioned information:

1. First vector of m personal characteristics (PC):

$${\vec{PC}}^{( j )} = 〈 {PC}_{1}^{( j )} , {PC}_{2}^{( j )} , {PC}_{3}^{( j )} , … {PC}_{m}^{( j )} 〉$$

2. Second vector of k behavioral categories (BC):

$${\vec{LDV}}^{( j )} = 〈 {BC}_{1}^{( j )} , {BC}_{2}^{( j )} , {BC}_{3}^{( j )} , … {BC}_{k}^{( j )} 〉$$

3. Third vector of p subjective wellbeing (SWB) measures:

$${\vec{SWB}}^{( j )} = 〈 {SWB}_{1}^{( i )} , {SWB}_{2}^{( j )} {SWB}_{3}^{( j )} , … {SWB}_{p}^{( j )} 〉$$

### Quantifying Exhibited Personal Characteristics

The exhibited personal characteristics represent a dynamic measure and are a function of a user's observed activities and behavior. The observed behavior is called “exhibited personal characteristics” because they are inferred through activities and behavior that a user exhibits and that correspond to a specific quantified profile of personal characteristics. The novel method categorizes the exhibited personal characteristics (PC) as a function of the lifestyle distribution vector (LDV) as follows:

$${\vec{PC}}_{exhibited}^{( j )} = 〈 {PC}_{map , 1}^{( j )} = {PC}_{map , 2}^{( j )} , {PC}_{map , 3}^{( j )} , … {PC}_{map , m}^{( j )} 〉 = f \{ {\vec{LDV}}^{( j )} \}$$

where PC^{(j)}_{map,1}, PC^{(j)}_{map,2}, PC^{(l)}_{map,3}, . . . PC^{(j)}_{map,m} represent m mapped personal characteristics scores obtained as a function of observed activities and behavior of user j, namely \vec{LDV}^{(j)}. “f” represents the function that maps the behavior and activity characteristics to personal characteristics.

To calculate the m exhibited personal characteristics, the user's behavior and activity distribution matrix (also called the LDM matrix) is first determined by concatenating the k-dimension second vector with the k-dimension second vectors of other users stored in the database. Personal information can be used from all users who have used the novel method and system in the past and have consented to have their data used and/or users who continue using the method and system and whose data until a given point of time can be used).

The system then derives or calculates a correlation matrix C by correlating the m-dimension first vector with the k-dimension second vector. The correlation matrix C thus contains correlations between behavior and personal characteristics. The matrix can be obtained by finding the most significant correlations between frequent behavior defined with \vec{LDV}^{(j)} and \vec{PC}^{(j)}. The matrix can also be obtained using theoretical correlations disclosed in psychological literature, such as Goldberg, L. R., “Then a miracle occurs: Focusing on behavior in social psychological theory and research,” Chapter 11 entitled, “Personality, Demographics, and Self-Reported Behavioral Acts: The Development of Avocational Interest Scales from Estimates of the Amount of Time Spent in Interest-Related Activities,” Oxford University Press 2010, pp. 205-226 ISBN 13:9780195377798. The correlation matrix C together with the behavior and activity distribution (LDM) matrix is used to calculate a weight (or effect) matrix W, described as:

W=LDM×C

where x is the matrix multiplication operator, LDM is a matrix of dimension N*k, C is a matrix of dimension k*m, N is the number of users stored in the database, and W is a matrix of dimensions N*m.

Each row of W corresponds to the weights (or the effect) of activity on exhibited personal characteristics, given by

$${\vec{w}}^{( j )} = 〈 {w}_{1}^{( j )} , {w}_{2}^{( j )} , {w}_{3}^{( j )} , … {w}_{m}^{( j )} 〉 , {\vec{w}}^{( j )}$$

which represents the change above or below the median personal characteristics that is exhibited by a user's lifestyle distribution vector (LDV). Thus, the exhibited personal characteristics of each user are obtained as the sum of the median personal characteristics, \vec{PC}_{med} and the Hadamard product (⋅) of user weights with the median personal characteristics as follows:

$${\vec{PC}}_{exhibited}^{( j )} = {\vec{PC}}_{med} + {\vec{PC}}_{med} \cdot {\vec{w}}^{( j )} = {\vec{PC}}_{med} ( 1 + {\vec{w}}^{( j )} )$$

Other embodiments of the novel method can use other mathematical models to compute PC_{exhibited}. Thus, the method calculates the miss-alignment parameter or ‘delta’ \vec{Δ}^{(j)} between an user's exhibited behavior (using \vec{PC}^{(j)}_{exhibited}) and the user's personal characteristics \vec{PC}^{(j)} as:

$${\vec{Δ}}^{( j )} = \vert {\vec{P C}}_{exhibited}^{( j )} - {\vec{P C}}^{( j )} \vert$$

where |⋅| is the absolute value function. The \vec{Δ}^{(j)} vector represents how miss-aligned an user's behavior is with respect to each of his/her personal characteristics. Thus, the greater the value of a component in \vec{Δ}^{(j)}, the more the user behaves away from this component. Because each component of \vec{Δ}^{(j)} is orthogonal, the cumulative Δ^{(j)} is given as the Euclidean norm of \vec{Δ}^{(j)}, i.e.,

$${Δ}^{( j )} = ‖ {\vec{Δ}}^{( j )} ‖ = \sqrt{{( {Δ}_{1}^{( j )} )}^{2} + {( {Δ}_{2}^{( j )} )}^{2} + {( {Δ}_{3}^{( j )} )}^{2} … {( {Δ}_{m}^{( j )} )}^{2}}$$

### Finding a Reference Group and Establishing the Recommendation

For each user x, a group of “role model” users (called the reference group) is defined. Each of these role model users has a higher cumulative subjective wellbeing SWB^{(j)} than does user x (i.e., higher third vector) and a lower mismatch in their lifestyles, Δ^{(j)} (i.e., lower miss-alignment parameter) than user x. Thus, for the user x, the reference group RG^{(x)} is expressed as:

RG^{(x)}={∀j∈(SWB^{(x)}<SWB^{(j)})Ω(Δ^{(x)}>Δ^{(j)})}

All users who are part of the set RG^{(x)} are now compared to user x in two possible ways: (1) the similarity in personal characteristics of user x to the reference group, and (2) the similarity in personal characteristics of user x to the exhibited personal characteristics of the reference group.

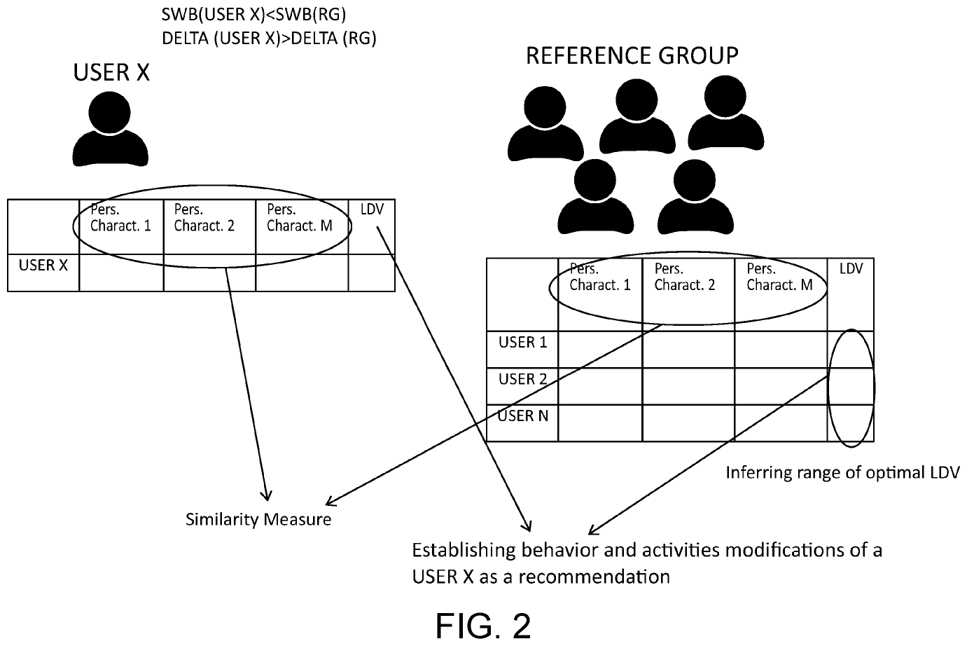

1. Similarity in personal characteristics of user x to the reference group. FIG. 2 illustrates this alternative, in which the personal characteristics of user x (\vec{PC}^{(x)}) are compared to the exhibited personal characteristics of each user j (\vec{PC}^{(j)}) in the set RG^{(x)} using a cosine similarity measure. This computation determines all the users that have similar static inherent personal characteristics as the user x, but who have higher SWB and lower Δ. The greater the cosine similarity value, the higher is the similarity between the two measures being compared. For a user y in RG^{(x)} the cosine similarity sim(x,y) between the users x and y can be expressed as:

$$sim ( x , y ) = \frac{{\sum }_{m} ( {PC}_{m}^{( x )} - {\overline{PC}}^{( x )} ) ( {PC}_{m}^{( y )} - {\overline{PC}}^{( y )} )}{\sqrt{{\sum }_{m} {( P {C}_{m}^{( x )} - {\overline{PC}}^{( x )} )}^{2} {\sum }_{m} {( P {C}_{m}^{( y )} - {\overline{PC}}^{( y )} )}^{2}}}$$

where PCₘ^{(x)} is the m personal characteristic of user x, P̅C̅^{(x)} is the average value of all the personal characteristics of user x, while PCₘ^{(y)} and P̅C̅^{(y)} represent the same values but for user y. This first alternative of the method uses the combination of dynamic filtering criteria based on Δ and the calculation of sim(x,y) based on static personal characteristics, and subsequently identifies users having the M highest values of sim(x,y) to be deemed the best users to help user x improve his/her activities and behavior. This is represented in FIG. 2.

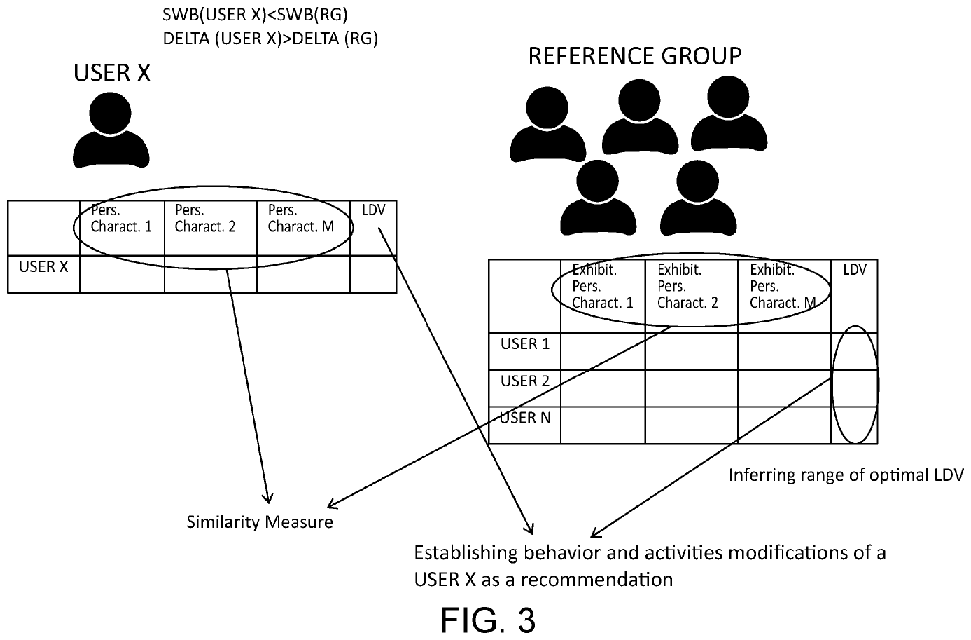

2. Similarity in personal characteristics of user x and exhibited personal characteristics of the reference group. FIG. 3 illustrates this alternative, in which the personal characteristics of user x (\vec{PC}^{(x)}) are compared to the exhibited personal characteristics of each user j (\vec{PC}^{(j)}_{exhibited}) in the set RG^{(x)} using the cosine similarity measure. This computation determines all the users having dynamic behavior that is well in line with the static personal characteristics of user x, but who have a higher SWB and a lower A. As defined above, the greater the cosine similarity value, the higher is the similarity between the two measures being compared. For a user y in RG^{(x)} the cosine similarity sim(x,y) between the users x and y is defined as:

$$sim ( x , y ) = \frac{{\sum }_{m} ( {PC}_{m}^{( x )} - {\overline{PC}}^{( x )} ) ( P {C}_{exhibited \cdot m}^{( y )} - {\overline{PC}}_{exhibited}^{( y )} )}{\sqrt{{\sum }_{m} {( {PC}_{m}^{( x )} - {\overline{PC}}^{( x )} )}^{2} {\sum }_{m} {( {PC}_{exhibited , m}^{( y )} - {PC}_{exhibited}^{( y )} )}^{2}}}$$

where PCₘ^{(x)} and P̅C̅^{(x)} represent the same values as above, while PC^{(y)}_{exhibited,m} represents the m component of the exhibited personal characteristic, and P̅C̅^{(y)}_{exhibited} represents the average value of the exhibited personal characteristic for user y. This variation of the method uses the combination of dynamic filtering criteria based on and calculation of sim(x,y) based on exhibited personal characteristics (based on dynamic activities and behavior), and subsequently identifies users having the M highest values of sim(x,y) to be deemed the best users to help user x improve his/her activities and behavior.

Once the top M similar users are identified from either of the above two variations of the present invention, the user's optimal behavior and activity distribution vector is determined/computed using LDVs of the most similar users. Thus, the range of optimal LDV can be suggested to the user x. This recommendation will increase the user x′s subjective wellbeing score, while decreasing his/her miss-alignment score.

### Determining the Range of Behavior and Activities (Actual Lifestyle) That are Good or Bad for the User

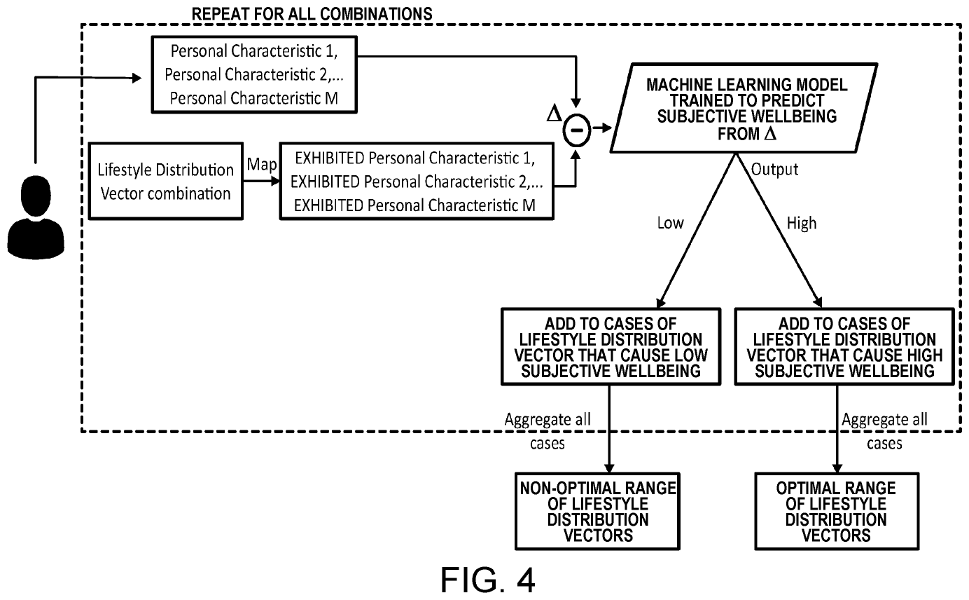

In case the number of users that form a part of the RG^{(x)} is low, another alternative can be used to provide recommendations to the user. FIG. 4 illustrates this alternative, in which behavior and activities that are either beneficial (good) or detrimental (bad) to the user's subjective wellbeing are determined. First, a machine learning model is trained to predict the subjective wellbring (SWB) score using \vec{Δ}. This is done using the information of all existing users. For a new user x, all possible combinations of the \vec{LDV}ᵢ^{(x)}, i∈(1, n) are generated, from which all resultant combinations n of \vec{PC}^{(x)}_{exhibited,i}, i∈(1, n) are obtained for the user. By comparing this to the user's \vec{PC}ᵢ^{(x)}, n, various possibilities \vec{Δ}ᵢ^{(x)}, i∈(1, n) are subsequently determined. Using the machine learning model built to predict SWB, those \vec{Δ}ᵢ^{(x)} that produce high wellbeing are identified. The \vec{LDV}ᵢ^{(x)} of all these cases are aggregated to obtain the range of optimal LDVs for user x. Similarly, the machine learning model can predict the cases for \vec{Δ}ᵢ^{(x)} that produce low wellbeing, and the \vec{LDV}ᵢ^{(x)} cases corresponding to this are aggregated to obtain the range of non-optimal LDVs for user x. Thus, the ranges of behavior that are good and bad for a user's subjective wellbeing are determined and can be provided as a recommendation.

In one particular embodiment, the novel method uses the personality measure using the BIG-5 personality inventory as the personal characteristics. The five personal characteristics are: extroversion, agreeableness, conscientiousness, (emotional) stability and intellect. Thus, for each user:

\vec{PC}^{(j)}=\vec{p}^{(j)}=<e^{(j)}, a^{(j)}, c^{(j)}, s^{(j)}, i^{(j)}>,

where e represents extrovision, a represents agreeableness, c represents conscientiousness, s represents stability, and i represents intellect.

Through the computational process described above for obtaining the exhibited personal characteristics from a person's exhibited lifestyle, the exhibited personality for each user is obtained as:

$${\vec{PC}}_{exhibited}^{( j )} = {\vec{p}}_{exhibited}^{( j )} = 〈 {e}_{map}^{( j )} , {a}_{map}^{( j )} , {c}_{map}^{( j )} , {s}_{map}^{( j )} , {i}_{map}^{( j )} 〉$$

Thus, the miss-alignment between a user's lifestyle and personality is expressed as:

$${Δ}^{( j )} = ‖ {\vec{Δ}}^{( j )} ‖ = ‖ {\vec{p}}_{exhibited}^{( j )} - {\vec{p}}^{( j )} ‖ = \sqrt{{( {e}^{( j )} - {e}_{map}^{( j )} )}^{2} + {( {a}^{( j )} - {a}_{map}^{( j )} )}^{2} + {( {c}^{( j )} - {c}_{map}^{( j )} )}^{2} + {( {s}^{( j )} - {s}_{map}^{( j )} )}^{2} + {( {i}^{( j )} - {i}_{map}^{( j )} )}^{2}}$$

The reference group determination and similarity measure calculation for the recommendation system are performed as described above, with 1≤m≤5. The ranges of behavior that are optimal and non-optimal can also be determined by using the machine learning model described previously.

The present invention has been described in particular detail with respect to specific possible embodiments. Those of skill in the art will appreciate that the invention may be practiced in other embodiments. For example, the nomenclature used for components, capitalization of component designations and terms, the attributes, data structures, or any other programming or structural aspect is not significant, mandatory, or limiting, and the mechanisms that implement the invention or its features can have various different names, formats, and/or protocols. Further, the system and/or functionality of the invention may be implemented via various combinations of software and hardware, as described, or entirely in software elements. Also, particular divisions of functionality between the various components described herein are merely exemplary, and not mandatory or significant. Consequently, functions performed by a single component may, in other embodiments, be performed by multiple components, and functions performed by multiple components may, in other embodiments, be performed by a single component.

Certain aspects of the present invention include process steps or operations and instructions described herein in an algorithmic and/or algorithmic-like form. It should be noted that the process steps and/or operations and instructions of the present invention can be embodied in software, firmware, and/or hardware, and when embodied in software, can be downloaded to reside on and be operated from different platforms used by real-time network operating systems. In the discussion above, certain aspects of one embodiment include process steps and/or operations and/or instructions described herein for illustrative purposes in a particular order, in particular the reception or capture of the first, second and third data. However, the particular order shown and discussed herein is illustrative only and not limiting. Those of skill in the art will recognize that other orders of the process steps are possible and, in some embodiments, one or more of the process steps discussed above can be combined. Consequently, the particular order discussed herein does not limit the scope of the invention as claimed.

1-14. (canceled)

15. A method for determining personalized parameters of a user, comprising:
receiving first data regarding personal characteristics of a user;
determining a first vector of personal characteristics of the user based on the first data;
receiving second data regarding behavior and activity characteristics of the user;
determining a second vector of behavior and activity characteristics of the user based on the second data;
receiving third data regarding subjective wellbeing measures of the user;
determining a third vector of subjective wellbeing measures of the user based on the third data;
determining exhibited personal characteristics of the user using the first vector and the second vector;
determining a miss-alignment parameter that compares the personal characteristics of the user to the first vector of the user;
identifying a reference group for the user, wherein the reference group includes a plurality of users each of whom has a higher third vector than that of the user and a lower miss-alignment parameter than that of the user;
determining a similarity measure between the user and the reference group, wherein the similarity measure identifies which of the users of the reference group are most similar to the user in terms of the personal characteristics and the behavior and activity characteristics; and
determining from the users of the reference group who are most similar to the user an optimal behavior and activity distribution vector for the user, and using the optimal behavior and activity distribution vector to recommend behavior and activity modifications to the user.

16. The method of claim 15, wherein the determining the exhibited personal characteristics of the user using the first vector and the second vector further comprises:
calculating a behavior and activity distribution matrix LDM of the user by concatenating the second vector of the user with second vectors of other users stored in a database;
calculating a correlation matrix C by correlating the first vector with the second vector;
calculating a weight matrix according to the equation:
W=LDM×C,
where x indicates a matrix multiplication operator; and
calculating the exhibited personal characteristics according to the equation:
$${\vec{PC}}_{exhibited}^{( j )} = {\vec{PC}}_{med} + {\vec{PC}}_{med} \cdot {\vec{w}}^{( j )} = {\vec{PC}}_{med} ( 1 + {\vec{w}}^{( j )} )$$
where \vec{PC}_{med} is the median of the first vector, and (⋅) is the Hadamard product of the first vector with the weight of the exhibited personal characteristics of the user j.

17. The method of claim 15, wherein the determining the similarity measure involves comparing the first vector of the user to a first vector of each of the plurality of users of the reference group using a cosine similarity measure, and wherein the first vector of each of the plurality of users is calculated using personal characteristics of each user.

18. The method of claim 15, wherein the determining the similarity measure is performed by comparing the first vector of the user to the exhibited personal characteristics of each of the plurality of users of the reference group using a cosine similarity measure, wherein the exhibited personal characteristics of each of the plurality of users are calculated using personal characteristics and behavior and activity characteristics of each user.

19. The method of claim 15, wherein the first data is received from a database on which results of a personality survey completed by the user are stored.

20. The method of claim 19, wherein the first data is inferred using machine learning to analyze qualities of the user selected from the group consisting of: a neighborhood in which the user lives and a community to which the user belongs.

21. The method of claim 15, wherein the second data is received as a vector that quantifies a proportion of various daily activities of the user, and wherein each of the various daily activities is weighted with a specific percentage.

22. The method of claim 21, wherein the second data is acquired in a manner selected from the group consisting of: a self-report of the user, a passive sensor, a mobile phone, a wearable device, and an activity recognition system of the user.

23. The method of claim 15, wherein the third data is based on a physiological signal selected from the group consisting of: a body temperature signal, a heart rate signal, a voice signal, and a facial expression recognition signal.

24. A method for determining personalized parameters of a user, comprising:
receiving first data regarding personal characteristics of a user;
determining a first vector of personal characteristics of the user based on the first data;
receiving second data regarding behavior and activity characteristics of the user;
determining a second vector of behavior and activity characteristics of the user based on the second data;
receiving third data regarding subjective wellbeing measures of the user;
determining a third vector of subjective wellbeing measures of the user based on the third data;
determining exhibited personal characteristics of the user using the first vector and the second vector;
determining a miss-alignment parameter that compares the personal characteristics of the user to the first vector of the user;
identifying a reference group for the user, wherein the reference group includes a plurality of users each of whom has a higher third vector than that of the user and a lower miss-alignment parameter than that of the user;
determining a similarity measure between the user and the reference group, wherein the similarity measure identifies which of the users of the reference group are most similar to the user in terms of the personal characteristics and the behavior and activity characteristics;
determining ranges of behavior and activities that are good or bad for the user by training a machine learning model to predict a score of the subjective wellbeing measures using \vec{Δ}, wherein \vec{Δ} is calculated by generating all possible combinations of the second vector of behavior and activity characteristics, mapping the combinations to the exhibited personal characteristics of the user, and comparing the exhibited personal characteristics to the first vector of personal characteristics; and
using the ranges of behavior and activities to recommend behavior and activity modifications to the user.

25. The method of claim 24, wherein the determining the exhibited personal characteristics of the user using the first vector and the second vector further comprises:
calculating a behavior and activity distribution matrix LDM of the user by concatenating the second vector of the user with second vectors of other users stored in a database;
calculating a correlation matrix C by correlating the first vector with the second vector;
calculating a weight matrix according to the equation:
W=LDM×C,
where x indicates a matrix multiplication operator; and
calculating the exhibited personal characteristics according to the equation:
$${\vec{PC}}_{exhibited}^{( j )} = {\vec{PC}}_{med} + {\vec{PC}}_{med} \cdot {\vec{w}}^{( j )} = {\vec{PC}}_{med} ( 1 + {\vec{w}}^{( j )} )$$
where \vec{PC}_{med} is the median of the first vector, and (⋅) is the Hadamard product of the first vector with the weight of the exhibited personal characteristics of the user j.

26. The method of claim 24, wherein the determining the similarity measure involves comparing the first vector of the user to a first vector of each of the plurality of users of the reference group using a cosine similarity measure, and wherein the first vector of each of the plurality of users is calculated using personal characteristics of each user.

27. The method of claim 24, wherein the determining the similarity measure is performed by comparing the first vector of the user to the exhibited personal characteristics of each of the plurality of users of the reference group using a cosine similarity measure, wherein the exhibited personal characteristics of each of the plurality of users are calculated using personal characteristics and behavior and activity characteristics of each user.

28. The method of claim 24, wherein the first data is received from a database on which results of a personality survey completed by the user are stored.

29. The method of claim 24, wherein the second data is received as a vector that quantifies a proportion of various daily activities of the user, and wherein each of the various daily activities is weighted with a specific percentage.

30. A system for determining personalized parameters for a user, comprising:
a memory; and
a processor configured to:
receive first data regarding different personal characteristics of a user, and determine a first vector of personal characteristics of the user based on the first data;
receive second data regarding behavior and activity characteristics of the user, and determine a second vector of behavior and activity characteristics of the user based on the second data;
receive third data regarding one or more subjective wellbeing measures of the user, and determine a third vector of wellbeing measures of the user based on the third data;
determine exhibited personal characteristics of the user using the first vector and the second vector;
determine a miss-alignment parameter between the exhibited personal characteristics and the first vector;
identify a reference group for the user, wherein the reference group includes a plurality of users each of whom has a higher third vector than that of the user and a lower miss-alignment parameter than that of the user;
determine a similarity measure between the user and the reference group, wherein the similarity measure identifies which of the users of the reference group are most similar to the user in terms of the personal characteristics and the behavior and activity characteristics; and
determine from the users of the reference group who are most similar to the user an optimal behavior and activity distribution vector for the user, and use the optimal behavior and activity distribution vector to recommend behavior and activity modifications to the user.

31. The system of claim 30, further comprising:
a server configured to provide the second data to the processor in a form selected from the group consisting of: a self-report of the user, an output from a passive sensor, an output from a mobile phone, an output from a wearable device, and an output from an activity recognition system of the user.

32. The system of claim 30, further comprising:
a sensor configured to monitor physiological signals of the user, wherein the physiological signals are selected from the group consisting of: a body temperature signal, a heart rate signal, a voice signal, and a facial expression recognition signal.

33. The system of claim 30, further comprising:
a server configured to provide the first data to the processor in a form of a personality survey completed by the user.
